(19) United States
(12) Patent Application Publication
(10) Pub. No.: US 2025/0366499 A1
(43) Pub. Date: Dec. 4, 2025

(54) Flavin-Containing Monoamine Oxidase MAO6sh Capable of Degrading Biogenic Amines and Application Thereof

(71) Applicants: Jiangnan University, Wuxi (CN); Jiangnan University (Shaoxing) Industrial Technology Research Institute, Shaoxing (CN)

(72) Inventors: Jian MAO, Wuxi (CN); Shuangping LIU, Wuxi (CN); Qilin YANG, Wuxi (CN)

(21) Appl. No.: 19/218,741

(22) Filed: May 27, 2025

(30) Foreign Application Priority Data
May 30, 2024 (CN) 202410688760X

(51) Int. Cl. A23L 5/20; A23L 13/40; A23L 27/50; C12H 1/00; C12J 1/00; C12N 9/06; C12N 15/70

(52) U.S. Cl. CPC A23L 5/25; A23L 13/48; A23L 27/50; C12H 1/003; C12J 1/00; C12N 9/0022; C12N 15/70; C12Y 104/03004; C12N 2800/101

(57) ABSTRACT

The present disclosure discloses a flavin-containing monoamine oxidase MAO6^{sh} capable of degrading biogenic amines and an application thereof, belonging to the technical field of molecular biology. The present disclosure provides a monoamine oxidase derived from Saccharopolyspora hirsuta, and achieves the expression of the monoamine oxidase in Escherichia coli. The present disclosure further provides an application of the monoamine oxidase in degradation of biogenic amines. Tryptamine, phenylethylamine and cadaverine can be effectively degraded by adding the monoamine oxidase to commercially available Huangjiu, the degradation rate is 33.76% or above, and the safety of fermented food is further improved.

### REFERENCE TO SEQUENCE LISTING

The instant application contains a Sequence Listing in XML format as a file named “YGHY-2025-10-SEQ.xml”, created on May 23, 2025, of 8,673 bytes in size, and which is hereby incorporated by reference in its entirety.

### TECHNICAL FIELD

The present disclosure relates to a flavin-containing monoamine oxidase MAO6^{sh} capable of degrading biogenic amines and an application thereof, belonging to the technical field of molecular biology.

### BACKGROUND

Biogenic amines are a general term for a class of amino basic organic compounds, are generally generated by decarboxylation reactions of amino acids, are commonly found in various fermented foods, and may cause symptoms such as diarrhea and vomiting after excessive intake. Research has shown that biogenic amines have a synergistic effect with ethanol, exacerbating the adverse effects after drinking. The main types of biogenic amines include putrescine (PUT), tyramine (TYR), histamine (HIS), cadaverine (CAD), phenylethylamine (PHE), tryptamine (TRY), spermine (SPE), and spermidine (SPD). At present, there is no clear regulation on the limit of alcoholic biogenic amines in China. Research suggests that a total content of biogenic amines in the fermented food should be less than 200 mg/kg, the upper limit of the content of histamine in an alcoholic beverage is 2 mg/L, and the upper limit of the content of tyramine is 10 mg/L. Therefore, strict control of the content of biogenic amines in the fermented food has become particularly crucial.

At present, the commonly used means for controlling biogenic amines is mainly implemented by controlling product production temperature, pH value and salt concentration or using irradiation and other methods. However, this means may affect the product quality to a certain extent. Using strains with relatively low activity of biogenic amine decarboxylase or strains with activity of biogenic amine oxidase as fermentation agents to ferment food can control the content of biogenic amines in the fermented food.

Microorganism-derived amine oxidases play important roles in degrading biogenic amines in food and ensuring food safety. Flavin-containing monoamine oxidases (EC 1.4.3.4) with FAD as a prosthetic group under amine oxidase branches are a type of enzymes existing in many microorganisms, and play an important role in a metabolic process of biogenic amines. These enzymes have an ability to oxidize and remove amino groups from biogenic amines, thereby transforming the biogenic amines to corresponding aldehydes. The substrate specificity of amine oxidases is relatively strong. At present, there is a lack of research on monoamine oxidases for degrading common biogenic amines in fermented food systems. To solve this critical issue, obtaining monoamine oxidases from microorganisms derived from fermented foods helps develop a strategy for enzymatic degradation of biogenic amines to rationally regulate and reduce the content of biogenic amines, which is of great significance for improving the quality of fermented foods such as Huangjiu (Chinese rice wine).

### SUMMARY

The present disclosure aims to solve the problem of generally higher content of biogenic amines in the existing traditional fermented foods, provide a flavin-containing monoamine oxidase capable of degrading biogenic amines derived from Saccharopolyspora hirsuta, and degrade biogenic amines in fermented foods by the flavin-containing monoamine oxidase to improve the quality of the traditional fermented foods.

The present disclosure provides a flavin-containing monoamine oxidase capable of degrading biogenic amines, shown in (a) or (b):
(a) a protein composed of an amino acid sequence shown in SEQ ID NO. 1; and
(b) a protein derived from (a) subjected to substitution, deletion, or addition of one or several amino acids in the amino acid sequence in (a) and having monoamine oxidase activity.

The present disclosure further provides a gene MAO6^{sh} encoding the monoamine oxidase.

In an embodiment, the gene contains a nucleotide sequence shown in SEQ ID NO. 3.

The present disclosure further provides recombinant expression plasmids carrying the gene.

In an embodiment, the plasmids include but are not limited to pET series, Duet series, pGEX series, pHY300, pHY300PLK, pPIC3K, pPIC9K or pTrc series vectors.

In an embodiment, the pET series vectors include pET24a(+), pET28a(+), pET29a(+), and pET30a(+); the Duet series vectors include pRSFDuet-1 and pCDFDuet-1; and the pTrc series vectors include pTrc99a.

In an embodiment, the recombinant expression plasmid is pET28a(+).

In an embodiment, the recombinant expression plasmid is linked to the nucleotide sequence shown in SEQ ID NO. 3 on pET28a(+).

The present disclosure further provides recombinant microbial cells expressing the monoamine oxidase.

In an embodiment, the recombinant microbial cells include but are not limited to Escherichia coli, Bacillus, or yeast.

The present disclosure further provides a genetically engineered bacterium using E. coli as a host to express the gene of the monoamine oxidase shown in SEQ ID NO. 3.

In an embodiment, the genetically engineered bacterium uses E. coli BL21 (DE3) as a host.

In an embodiment, the genetically engineered bacterium uses pET series plasmids as expression vectors.

In an embodiment, the genetically engineered bacterium uses pET28a(+) as an expression vector to express the monoamine oxidase shown in SEQ ID NO. 3.

The present disclosure further provides a method for constructing the genetically engineered bacterium, which involves ligating a gene sequence shown in SEQ ID NO. 3 into a vector, followed by transformation into E. coli cells.

In an embodiment, the vector is pET28a(+).

In an embodiment, the gene sequence is linked between NheI and HindIII sites of pET28a(+).

The present disclosure further provides a method for producing the monoamine oxidase. The method involves culturing the genetically engineered bacterium in a culture medium for a period of time, and collecting the monoamine oxidase.

In an embodiment, the method involves collecting bacterial cells from a cell culture fluid, and crushing the cells to obtain a crude enzyme solution containing the monoamine oxidase.

In an embodiment, the method further involves purifying the crude enzyme solution.

In an embodiment, the purification includes but is not limited to affinity chromatography, gel filtration chromatography/molecular sieve, ion exchange chromatography, ammonium sulfate precipitation/polyethylene glycol (PEG) precipitation, and other purification methods well known in the art.

In an embodiment, the affinity chromatography includes metal chelate affinity chromatography (such as purifying proteins using His tags), immunoaffinity chromatography, etc.

In an embodiment, the purification is carried out using nickel column affinity chromatography.

In an embodiment, the culture involves inoculating the genetically engineered bacterium into an LB culture medium for culturing, and inducing enzyme production with IPTG when the OD600 is 0.6-0.8.

In an embodiment, the induction is carried out at 16-37° C.

In an embodiment, the final concentration of the IPTG is 0.4-0.6 mmol·L⁻¹.

In an embodiment, the induction is carried out at 16° C. and 200 rpm for 16 h.

The present disclosure further provides a method for reducing biogenic amines in foods, which involves adding the monoamine oxidase shown in SEQ ID NO. 1 to the foods to degrade biogenic amines in the foods.

In an embodiment, the method involves contacting the monoamine oxidase with biogenic amines in the environment.

In an embodiment, the environment includes liquid environment, semi-solid environment, or solid environment.

In an embodiment, the method includes reducing the content of biogenic amines in fermented foods.

In an embodiment, the fermented foods include fermented dairy products, fermented bean products, fermented meat products, fermented cereal products, fermented vegetable products, fermented seasonings, or fermented alcoholic beverages.

In an embodiment, the fermented dairy products include but are not limited to yogurt or cheese; and the cheese includes but is not limited to cheddar cheese, mozzarella cheese, goat cheese, etc.

In an embodiment, the fermented meat products include but are not limited to sausages (such as German sausages and Italian salami), ham, bacon, dried meat, and fish sauce.

In an embodiment, the fermented vegetable products include but are not limited to pickled vegetables, fermented cabbage, kimchi, pickled vegetables with sauce, etc.

In an embodiment, the fermented bean products include but are not limited to fermented bean curd or natto.

In an embodiment, the fermented seasonings include but are not limited to soy sauce, miso, vinegar, and broad bean paste.

In an embodiment, the fermented alcoholic beverages include but are not limited to beer, wine, Huangjiu, and rice wine.

In an embodiment, the application involves adding the monoamine oxidase to the Huangjiu to reduce the content of main biogenic amines therein.

In an embodiment, the application involves adding the recombinant monoamine oxidase to the Huangjiu, followed by reaction at 25-28° C. for 24-48 h.

In an embodiment, the biogenic amine includes but is not limited to one or more of tryptamine, phenylethylamine, putrescine, cadaverine, histamine, tyramine, spermidine, and spermine.

The present disclosure provides an enzyme preparation for degrading biogenic amines. The enzyme preparation contains a monoamine oxidase with an amino acid sequence shown in SEQ ID NO. 1.

In an embodiment, the enzyme preparation further contains stabilizers for protecting the stability of enzymes during production, storage and use to prevent enzyme inactivation or degradation.

In an embodiment, the stabilizers include but are not limited to saccharides, polyols, proteins, polymers, metal ions, etc.

The present disclosure provides a method for degrading biogenic amines in an environmental system. The method involves using the monoamine oxidase or the genetically engineered bacterium expressing the monoamine oxidase to degrade biogenic amines. The amino acid sequence of the monoamine oxidase is shown in SEQ ID NO. 1. The environmental system is non-biological internal environment.

In an embodiment, the environmental system includes but is not limited to dairy products, fish products, meat products, and fermented foods.

In an embodiment, the environmental system includes but is not limited to fermented foods such as Huangjiu, soy sauce, vinegar, low-salt soy sauce, cooking wine, wine, shrimp sauce, and fish sauce.

In an embodiment, the genetically engineered bacterium uses bacteria or fungi as host cells.

In an embodiment, the biogenic amine includes but is not limited to one or more of tryptamine, phenylethylamine, putrescine, cadaverine, histamine, tyramine, spermidine, and spermine.

The present disclosure further provides an application of the above-mentioned enzyme preparation, or the above-mentioned monoamine oxidase with an amino acid sequence shown in SEQ ID NO. 1, or the engineered bacterium expressing the monoamine oxidase with an amino acid sequence shown in SEQ ID NO. 1 in the preparation of products for degrading biogenic amines in environmental systems.

The present disclosure further provides an application of the above-mentioned enzyme preparation, or the above-mentioned monoamine oxidase with an amino acid sequence shown in SEQ ID NO. 1, or the genetically engineered bacterium expressing the monoamine oxidase with an amino acid sequence shown in SEQ ID NO. 1 in the preparation of fermented foods.

In an embodiment, the fermented foods include but are not limited to fermented vegetables and alcoholic beverages.

In an embodiment, the fermented foods include but are not limited to Huangjiu, soy sauce, vinegar, low-salt soy sauce, cooking wine, wine, shrimp sauce, and fish sauce.

In an embodiment, the genetically engineered bacterium uses bacteria or fungi as host cells.

### Beneficial Effects:

(1) The monoamine oxidase MAO6^{sh} from Saccharopolyspora hirsuta provided by the present disclosure has relatively strong performance in degrading biogenic amines. This enzyme also has certain pH stability and temperature stability, and can tolerate a certain concentration of ethanol, thereby helping to achieve the degradation of biogenic amines in alcoholic beverages.
(2) The present disclosure also constructs recombinant E. coli BL21 (DE3) expressing the monoamine oxidase MAO6^{sh}. The recombinant E. coli BL21 (DE3) is cultured at 16° C. for 16 h and subjected to induction culture in an TB culture medium, and then, every 100 ml of bacterial solution with the OD600 of 1 contains 17.5 mg of MAO6^{sh} enzyme protein.
(3) The present disclosure further provides an application of the monoamine oxidase MAO6^{sh} in the aspect of degrading biogenic amines in fermented foods. This enzyme can degrade tryptamine, putrescine, cadaverine, phenylethylamine, histamine, tyramine, histamine, spermidine, and spermine in 18% vol of commercially available Huangjiu within 48 h at degradation rates of 54.06%, 33.76%, 43.78%, 5.78%, 21.44%, 19.31%, 8.51%, and 7.75% respectively. This enzyme particularly reflects the degradation potential for tryptamine, putrescine and cadaverine, thereby helping to further improve the safety of fermented foods.

### BRIEF DESCRIPTION OF FIGURES

FIG. 1 shows an agarose gel electrophoresis verification diagram of a monoamine oxidase gene; M: DNA Maker; 1: PCR amplification product (1302 bp).

FIG. 2 shows a plasmid profile of an expression vector of a monoamine oxidase pET28a-MAO6^{sh}.

FIG. 3 shows an electrophoretogram of protein separation and purification processes of a recombinant monoamine oxidase MAO6^{sh}; M: Unstained Protein Ladder; 1: pET28a-empty expression vector; 2. crude enzyme solution supernatant of monoamine oxidase pET28a-MAO6^{sh} induced at 16° C.; 3. crude enzyme solution supernatant of monoamine oxidase pET28a-MAO6^{sh} induced at 20° C.; 4. crude enzyme solution supernatant of monoamine oxidase pET28a-MAO6^{sh} induced at 28° C.; 5: crude enzyme solution supernatant of monoamine oxidase pET28a-MAO6^{sh} induced at 37° C.; 6: purified enzyme solution of monoamine oxidase pET28a-MAO6^{sh} induced at 16° C.; 7. crude enzyme solution precipitate of monoamine oxidase pET28a-MAO6^{sh} induced at 16° C.; 8: crude enzyme solution precipitate of monoamine oxidase pET28a-MAO6^{sh} induced at 20° C.; 9: crude enzyme solution precipitate of monoamine oxidase pET28a-MAO6^{sh} induced at 37° C.

FIG. 4 shows the ability of a monoamine oxidase MAO6^{sh} for degrading different biogenic amines before and after purification. Putrescine (PUT), tyramine (TYR), histamine (HIS), cadaverine (CAD), phenylethylamine (PHE), tryptamine (TRY), spermine (SPE), and spermidine (SPD).

FIG. 5 shows the relative enzyme activity of a monoamine oxidase MAO6^{sh} at different temperatures. Putrescine (PUT), tyramine (TYR), histamine (HIS), cadaverine (CAD), phenylethylamine (PHE), tryptamine (TRY), spermine (SPE), and spermidine (SPD).

FIG. 5 and FIG. 6 show the temperature stability of a monoamine oxidase MAO6^{sh}. Putrescine (PUT), tyramine (TYR), histamine (HIS), cadaverine (CAD), phenylethylamine (PHE), tryptamine (TRY), spermine (SPE), and spermidine (SPD).

FIG. 7 shows the relative enzyme activity of a monoamine oxidase MAO6^{sh} at different pH values. Putrescine (PUT), tyramine (TYR), histamine (HIS), cadaverine (CAD), phenylethylamine (PHE), tryptamine (TRY), spermine (SPE), and spermidine (SPD).

FIG. 8 shows the pH stability of a monoamine oxidase MAO6^{sh}. Putrescine (PUT), tyramine (TYR), histamine (HIS), cadaverine (CAD), phenylethylamine (PHE), tryptamine (TRY), spermine (SPE), and spermidine (SPD).

FIG. 9 shows the relative enzyme activity of a monoamine oxidase MAO6^{sh} under different ethanol conditions. Putrescine (PUT), cadaverine (CAD), and tryptamine (TRY).

FIG. 10 shows a phylogenetic tree diagram of a monoamine oxidase gene MAO6^{sh}

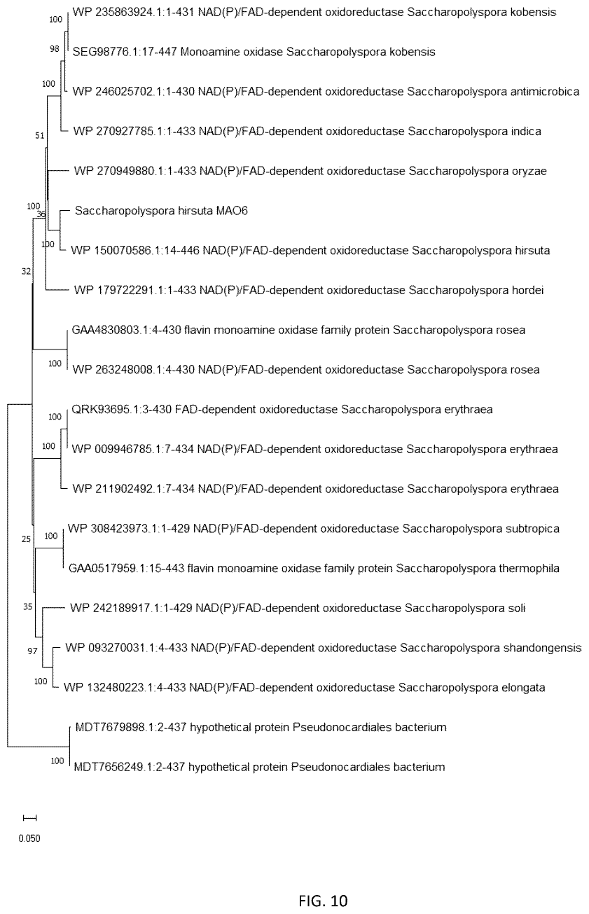

### DETAILED DESCRIPTION

### (1) Technical Terms:

The “dairy products” involved in the present disclosure refer to foods made from animal milk as a raw material through different processing methods, and the types include but are not limited to pasteurized milk, sterilized milk, blended milk, fermented milk, whole milk powder, skimmed milk powder, whole milk powder with sugar, flavored milk powder, infant milk powder and other formula milk powder, condensed milk, milk fats, cheeses, ice creams, casein, milk slices, lactose, etc.

The “fish products” involved in the present disclosure refer to products made from fish meat or certain organs of fish as main raw materials through various processing methods. The processing methods include pickling, fumigating, drying, freezing, canning, fermentation, etc.

The “meat products” involved in the present disclosure refer to products made from animal muscle tissues or edible internal organs as main raw materials through various processing methods. According to the national standard for meat and meat product terms (GB/T 19480-2009), meat products are divided into two categories: Chinese style meat products and Western style meat products. The Chinese style meat products include cured meat, corned meat, Chinese ham, dried meat floss, dried meat dice, dried meat slice, stewed meat in seasoning, meat flavored with fermented rice, smoked meat products, Chinese sausage, cured sausage, air-dried sausage, fresh sausage products, smoked and fresh sausage, semi-dry sausage, dry sausage, prepared meat products, meat cake, and salted meat. The Western style meat products include cooked and smoked ham, cooked and smoked sausage, sausage products, blood sausage, fermented sausage, bacon, ham or meat sausage.

The “fermented foods” involved in the present disclosure refer to foods made by fermentation of microorganisms (such as bacteria, yeast, or fungi). The fermented foods include but are not limited to alcoholic beverages, fish and shrimp sauce, fruit wine, soy sauce, yogurt, cheese, fermented glutinous rice, pickled vegetables, soy sauce, table vinegar, fermented soy beans, Huangjiu, beer, wine, etc.

The “biogenic amines” involved in the present disclosure refer to low-molecular-weight organic compounds having biological activity and containing amino groups, including but not limited to tryptamine (TRY), phenylethylamine (PHE), putrescine (PUT), cadaverine (CAD), histamine (HIS), tyramine (TYR), spermidine (SPD), and spermine (SPE).

### (2) Reagent

The Huangjiu and other products involved in the following examples were purchased from a supermarket in Wuxi, Jiangsu Province.

### (3) Culture Medium

An LB culture medium was formed by 5 g/L yeast extract, 10 g/L tryptone and 10 g/L sodium chloride by adjusting the pH to 7.0 with NaOH, and was sterilized under a high pressure at 121° C. for 10 min.

A TB culture medium was purchased from Qingdao Hopebio company, and was sterilized under a high pressure at 121° C. for 10 min.

### (4) Detection Method

The content of biogenic amines was detected by high-performance liquid chromatography (HPLC).

Enzyme activity measurement: The activity of a biogenic amine oxidase was measured by an indirect measurement method of catalase. The amine oxidase acts on biogenic amines to degrade the biogenic amines into corresponding aldehydes and hydrogen peroxide. Under the presence of peroxidase, hydrogen peroxide reacts with 4-aminoantipyrine and 2,4,6-tribromo-3-hydroxybenzoic acid to generate a quinone dye. The product has a maximum absorption value at 510 nm, and the magnitude of the activity of the amine oxidase was linearly related to the color intensity of the product within a certain range, so that the activity of the amine oxidase can be measured by measuring the change in A510.

The reaction was carried out in a 96-well plate. The reaction system included 20 μl of enzyme solution (80 ug·mL⁻¹) and 100 μl of prepared solution (including 200 mmol·L⁻¹ potassium phosphate buffer with a pH of 7.6, 1.5 mmol·L⁻¹ 4-aminoantipyrine, and 1 mmol·L⁻¹ 2,4,6-tribromo-3-hydroxybenzoic acid). To initiate the reaction, 20 μL of biogenic amine solution (10 mmol·L⁻¹) and 70 L of peroxidase (1.4 mg·mL⁻¹) were added, the absorbance was measured at 510 nm, the reaction temperature was 37° C., and the reaction time was 10 min. The change in absorbance of 0.01 per minute was defined as one enzyme activity unit (U).

Definition of specific activity of monoamine oxidase (U/mg): The enzyme activity per milligram of protein.

### Example 1

### PCR Amplification of Monoamine Oxidase Gene MAO6^{sh}

Specific steps were as follows:
(1) Primers were designed according to the monoamine oxidase gene (WP_150070586.1) in Saccharopolyspora hirsuta in an NCBI database, and the DNA of Saccharopolyspora hirsuta T14 preserved in the laboratory was used as a template to amplify the monoamine oxidase gene MAO6^{sh}.

The primers required for amplification were as follows:

F:
5′-ctagctagcATGGACTCCTWCGACGTCGTSGTCA
TCGGTGCCGGrTTCGCCGG-3′;
and
R:
5′-CAAGCTTTCAGCCTCGSGGGCCGCGCAGGGCGTC
CTGCACGGCCCTGGAGGCGCGCAGGCCGCTC-3′.

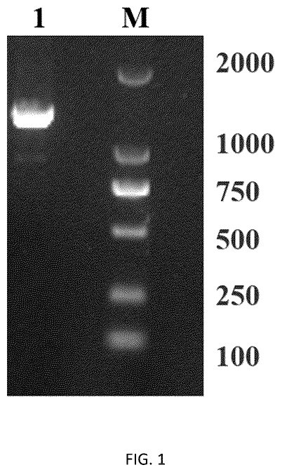

A PCR reaction solution was prepared according to the requirements of a reaction system of TaKaRa LA Taq. The PCR amplification system was as follows: pre-denaturation at 98° C. for 10 s, annealing at 75° C. for 30 s, and extension at 72° C. (1 min·kb⁻¹) for 35 cycles.
(2) The DNA of Saccharopolyspora hirsuta T14 was used as a template for PCR amplification, and the amplification results of PCR products were verified by 1.2% agarose gel electrophoresis. As shown in FIG. 1, the amplified sequence size was the same as the target gene sequence size, approximately 1302 bp, indicating successful amplification. The PCR products were purified and then sent to the company for sequencing, and the sequencing results were shown in SEQ ID NO. 2 (with enzyme digestion sites Nhe I and Hind III at both ends of the sequence).

### Example 2

### Construction of Genetically Engineered Bacterium Capable of Producing Monoamine Oxidase Gene MAO6^{sh}

The amine oxidase gene MAO6^{sh} amplified in Example 1 was linked to plasmids so as to be transformed to microbial cells to construct a genetically engineered bacterium capable of producing the monoamine oxidase gene MAO6^{sh}.

Optionally, the plasmids include but are not limited to pET series, Duet series, pGEX series, pHY300, pHY300PLK, pPIC3K, pPIC9K, or pTrc series vectors; the pET series vectors include pET24a(+), pET28a(+), pET29a(+), and pET30a(+); the Duet series vectors include pRSFDuet-1 and pCDFDuet-1; and the pTrc series vectors include pTrc99a.

Optionally, the host was a bacterial cell or a fungal cell, including but not limited to E. coli, Bacillus, or yeast.

Taking recombinant E. coli as an example, the construction process of the genetically engineered bacterium pET28a-MAO6^{sh} was described as follows:

### (1) Obtaining of Target Fragment

The DNA of the strain Saccharopolyspora hirsuta T14 preserved in the laboratory was used as a template to complete PCR amplification by combining primers and whole genome DNA together, and the PCR reaction system and the amplification procedure were the same as those described in Example 1. The gel of the PCR product with correctly verified bands was carefully cut, and the gel was recovered and purified to obtain the monoamine oxidase gene fragment MAO6^{sh}. After sequencing, the nucleotide sequence of the gene was determined to be as shown in SEQ ID NO. 3.

### (2) Enzyme Digestion and Linkage

The plasmid pET-28a(+) and the target fragment obtained in step (1) were subjected to double enzyme digestion respectively with restriction enzymes NheI and HindIII. The enzyme digestion system was as follows: 50 μL of plasmid, 2.5 μl of restriction enzyme Nhe I and 2.5 μL of restriction enzyme HindIII, and 5 μl of FastDigest Green Buffer. The components in the enzyme digestion system were fully mixed uniformly, and then, the mixture was placed in a metal bath at 37° C. for reacting for 60 min. After double enzyme digestion, the gene fragment and the plasmid were recovered and purified, and then mixed in a molar ratio of (3-10):1. An equal volume of Solution I ligase was added and fully mixed uniformly, and then, the mixture was incubated overnight in a metal bath at 16° C. to prepare a recombinant vector pET28a-MAO6^{sh}.

### (3) Transformation

After E. coli BL21 (DE3) competent cells preserved at −80° C. were placed in an ice bath for 10 min, 10 μL of linkage product to be transformed, obtained in step (2), was sucked with a pipette and added to the competent cells and gently blown and sucked and mixed uniformly, and then, the mixture was placed in an ice bath for 30 min. After the ice bath, the mixture was subjected to thermal shock at 42° C. for 45 s and then immediately taken out and placed in ice for 2 min. Then, 700 μL of LB liquid culture medium was added for shaking culture at 37° C. for 60 min at 200 r·min⁻¹. Centrifugation was carried out for 1 min at 8000 r·min⁻¹, most of the supernatant was discarded, and about 200 μL of supernatant was retained to resuspend the bacterial cells. The bacterial solution was uniformly coated on an LB solid culture medium plate containing 50 mg·L⁻¹ kanamycin, the plate was inverted and cultured overnight in an incubator at 37° C. until a single colony grows up and was picked, and then, PCR verification was carried out for screening positive transformants.

### (4) Enzyme Digestion Verification

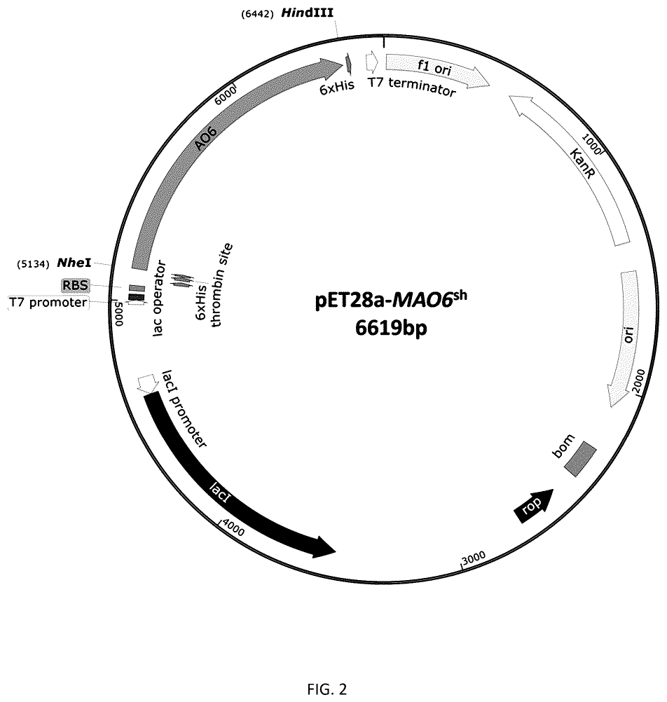

The plasmid of the recombinant bacterium was extracted and subjected to NheI and HindIII double enzyme digestion to respectively obtain a pET-28a(+) fragment of 5369 bp and a target fragment of 1302 bp. The target fragment was sent to the company for sequencing, and the sequencing results were consistent with the target gene sequence, verifying the successful construction of the recombinant bacterium E. coli BL21/pET28a-MAO6^{sh}. The recombinant enzyme expressed by this strain was named as MAO6^{sh}. The construction process was shown in FIG. 2.

### Example 3

### Induced Expression and Purification of Recombinant Enzyme MAO6^{sh}

Specific steps were as follows:
(1) The recombinant bacterium E. coli BL21/pET28a-MAO6^{sh} constructed in Example 2 was inoculated into an LB culture medium containing 50 mg·L⁻¹ kanamycin, and cultured at 37° C. for 14 h at 150 r·min⁻¹ to prepare a seed solution.
(2) The obtained seed solution was transferred into a TB fermentation culture medium containing 50 mg·L⁻¹ kanamycin at an inoculation volume of 5% (v/v), and cultured at 37° C. at 160 r·min⁻¹ until the OD600 was 0.4-0.6. The IPTG with a final concentration of 0.5 mmol·L⁻¹ was added and cultured at 16° C., 20° C., 28° C. and 37° C. respectively for 16 h at 160 rpm to obtain bacterial solutions.

The enzyme yield of the monoamine oxidase in the bacterial solution was measured, and the results showed that 17.5 mg of MAO6^{sh} enzyme protein was contained in every 100 mL of bacterial solution with the OD600 of 1 induced at 16° C.

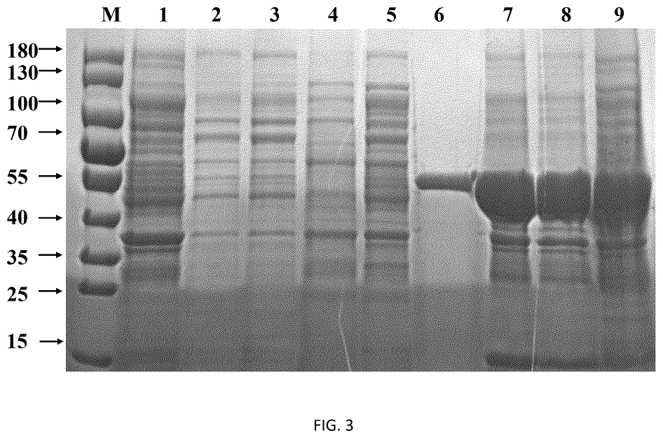

The activity of the monoamine oxidase was measured, and the results showed that at 16° C., the specific enzyme activities of the monoamine oxidase in the crude enzyme solution for tryptamine, putrescine and cadaverine were 13.94 U/mg, 15.66 U/mg and 14.70 U/mg respectively. When the induction temperature was slightly higher, more inclusion bodies were produced (as shown in FIG. 3), leading to a decrease in enzyme activity.
(3) The bacterial solutions were respectively centrifuged at 4° C. for 10 min at 12000 r·min⁻¹, and then, lower-layer bacterial cells were collected; and 0.1 mol·L⁻¹ phosphate buffer saline with a pH of 7.4 was added to resuspend the bacterial cells, and then, the bacterial cells were collected by centrifugation. The above-mentioned steps were repeated twice. An ultrasonic cell crusher was used to crush the bacterial cells under ultrasonic conditions of 400 W, working for 2 s at an interval of 3 s, and a crushing time of 1 h. After crushing, the supernatant and precipitate were collected by centrifugation at 4° C. at 12000 r·min⁻¹, filtered through a 0.22 μM filter membrane, and stored at a low temperature for future use.
(4) Purification of Recombinant Enzyme MAO6^{sh}

The supernatant obtained in step (3) was processed by an affinity

chromatography column HisTrap™ HP (GE Healthcare) to purify the protein, and an AKTA avant 25 instrument was used to separate and purify the target protein.

The supernatant of the crude enzyme solution before MAO6^{sh} purification and the protein after MAO6^{sh} purification were respectively analyzed by SDS-PAGE (sodium dodecyl sulfate-polyacrylamide gel electrophoresis), and the results were shown in FIG. 3. The molecular weight of the recombinant monoamine oxidase MAO6^{sh} was about 46.7 kDa. The crude enzyme solution was analyzed by SDS-PAGE, confirming the successful expression of the recombinant engineering bacterium. The lane 6 proves that the pure enzyme of the target protein was successfully obtained through the separation and purification of an affinity chromatography nickel column. Although induced by multiple conditions, when the recombinant bacterium E. coli BL21/pET28a-MAO6^{sh} expresses the enzyme gene, inclusion bodies easily occur. The inclusion body phenomenon can be observed in lanes 7, 8, and 9.

### Example 4

### Preparation of Enzyme Preparation of Monoamine Oxidase MAO6^{sh}

The monoamine oxidase MAO6^{sh} purified in Example 3 was mixed with an enzyme stabilizer to prepare an enzyme preparation containing the monoamine oxidase MAO6^{sh}. The stabilizer refers to a substance that can protect the stability of enzymes during production, storage and use to prevent enzyme inactivation or degradation, including but not limited to saccharides, polyols, proteins, polymers, metal ions, etc.

### Example 5

### Degradation of Different Biogenic Amines by Recombinant Enzyme MAO6^{sh}

The catalytic ability of the monoamine oxidase MAO6^{sh} before and after purification was measured respectively using a single biogenic amine as a substrate. Systems containing putrescine, tyramine, histamine, cadaverine, phenylethylamine, tryptamine, spermine or spermidine with a concentration of 10 mmol/L were prepared respectively, the pure enzyme in Example 3 was added to the reaction system after adjusting the protein concentration (80 ug/mL, 20 uL) for reacting for 30 min at 37° C., and the results were shown in

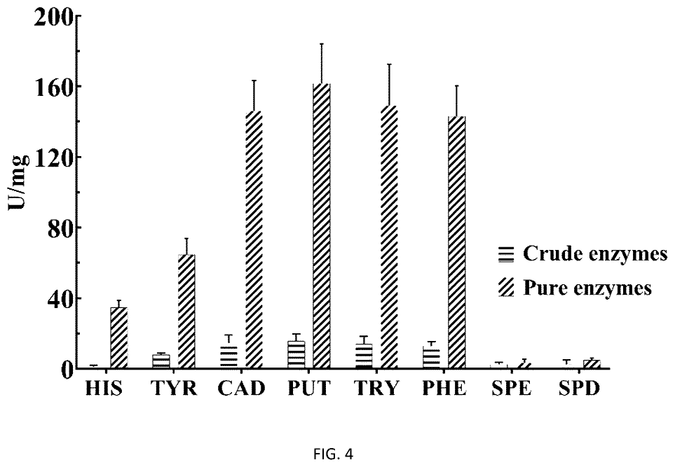

FIG. 4. The MAO6^{sh} has better effects on substrates, namely putrescine, tryptamine and cadaverine. The MAO6^{sh} has a specific enzyme activity of 161.59 U/mg for putrescine, 149.16 U/mg for tryptamine, and 146.05 U/mg for cadaverine.

### Example 6

### Degradation of Biogenic Amines by Recombinant Enzyme MAO6^{sh} at Different Reaction Temperatures, and Temperature Stability

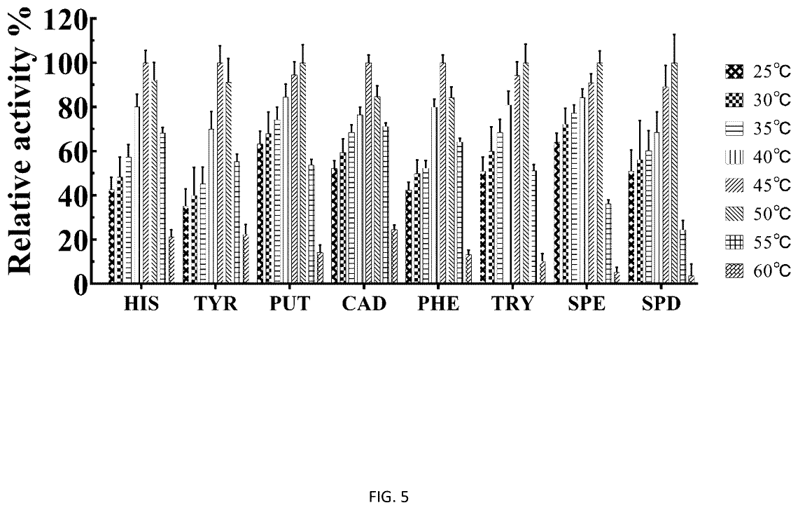

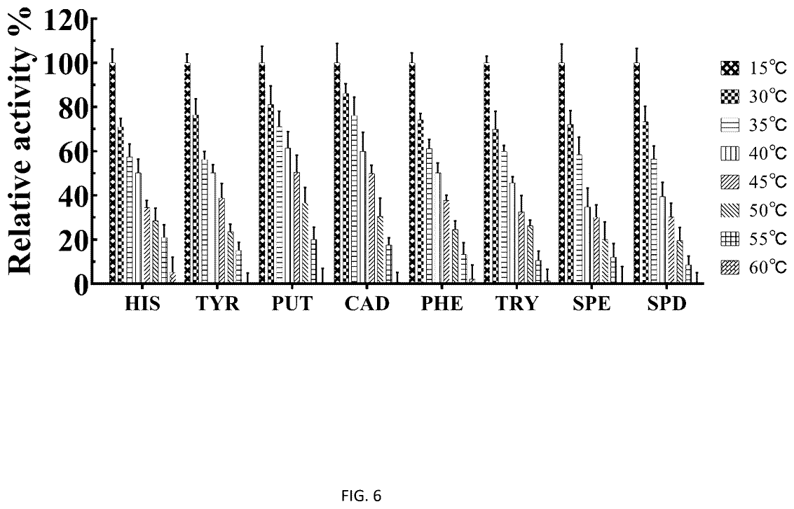

The reaction system was the same as that of Example 5, except that the temperature of the reaction system was changed to 25° C., 30° C., 35° C., 40° C., 45° C., 50° C., 55° C. and 60° C. respectively, the enzyme activity of the recombinant enzyme MAO6^{sh} under different temperature reaction conditions was measured, the relative enzyme activity at each temperature was calculated respectively using a single biogenic amine as a substrate at the highest enzyme activity of 100%, and the results were shown in FIG. 5. The optimal temperature for the MAO6^{sh} for the most suitable substrate was approximately 45-50° C. When the temperature was within the range of 25-45° C., the enzyme activity decreased rapidly with the increasing temperature.

After the enzyme solution purified in Example 3 was respectively incubated for 30 min in water baths at 15° C., 30° C., 35° C., 40° C., 45° C., 50° C., 55° C. and 60° C., the enzyme activity was measured according to the standard enzyme reaction system to determine the stable temperature range thereof, and the results were shown in FIG. 6. When the incubation temperature exceeds 45° C., about 30% of residual enzyme activity can be retained, indicating that the monoamine oxidase has certain heat resistance. When the incubation temperature was 50° C., the residual enzyme activity for putrescine was 36.66%, the residual enzyme activity for cadaverine was 30.55%, and the residual enzyme activity for tryptamine was 26.34%.

### Example 7

### Degradation of Biogenic Amines by Recombinant Enzyme MAO6^{sh} at Different Reaction pH Values, and pH Stability

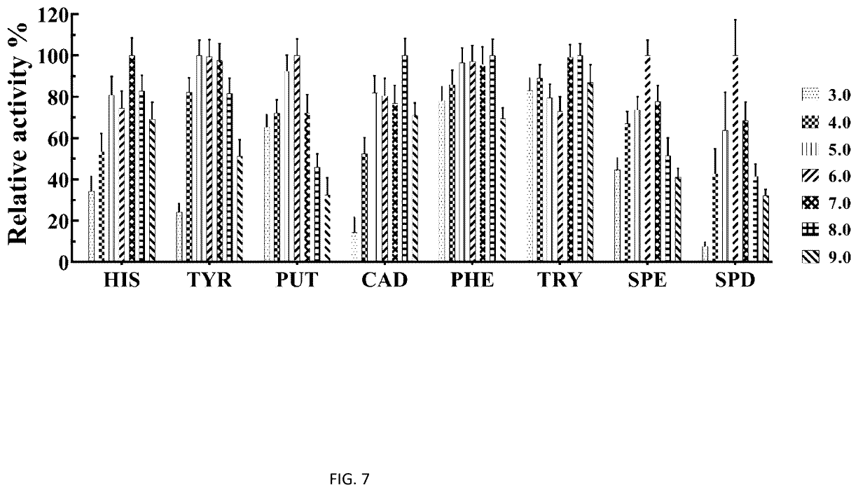

The reaction system was the same as that of Example 5, except that the pH of the reaction system was respectively adjusted to 3.0-9.0, the enzyme activity of the recombinant enzyme MAO6^{sh} under different pH reaction conditions was measured, the relative enzyme activity at each temperature was calculated respectively using a single biogenic amine as a substrate at the highest enzyme activity of 100%, and the results were shown in FIG. 7. When putrescine was used as a substrate, the optimal pH for the MAO6^{sh} was about 5.0-6.0. When the pH was within the range of 3.0-5.0, the enzyme activity decreased rapidly with the increase of the pH. However, under alkaline conditions, the tolerance was stronger than the acid tolerance.

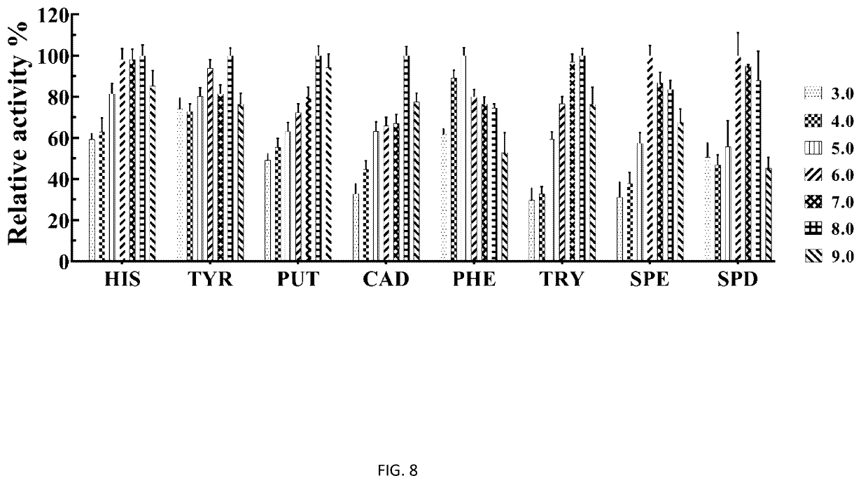

After the recombinant enzyme solution purified in Example 3 was diluted by an appropriate multiple, the enzyme was incubated at 45° C. for 30 min under different pH ambient conditions, the enzyme activity was measured, and the results were shown in FIG. 8. The monoamine oxidase MAO6^{sh} was more stable under neutral to alkaline conditions.

### Example 8

### Influence of Ethanol with Different Concentrations on Monoamine Oxidase MAO6^{sh}

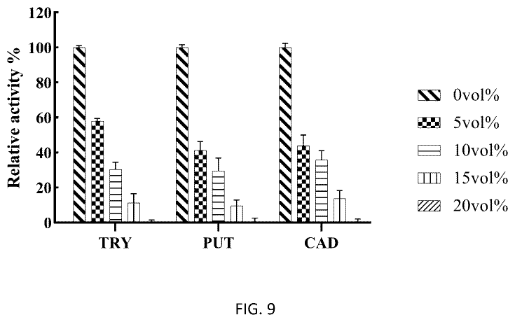

The reaction system was the same as that of Example 5, except that ethanol with different concentrations (0-20% vol) was added respectively. The results showed that the recombinant monoamine oxidase MAO6^{sh} inhibited the enzyme activity of each biogenic amine to different degrees. When the concentration of ethanol was lower than 10% vol, the relative enzyme activities of the monoamine oxidase MAO6^{sh} for putrescine (29.44%), tryptamine (30.27%) and cadaverine (35.78%) were about 30%. When the concentration of ethanol was about 15 vol %, the relative enzyme activities of the monoamine oxidase MAO6^{sh} for putrescine, tryptamine and cadaverine were 9.57%, 11.21% and 13.67% respectively (FIG. 9).

The above results indicate that the monoamine oxidase MAO6^{sh} has a good catalytic degradation ability for putrescine, tryptamine and cadaverine in a system containing a low-concentration ethanol solution. This characteristic provides a good foundation for the application in degradation of alcoholic beverages containing low-concentration ethanol (such as beer and Huangjiu) or systems rich in putrescine, tryptamine and cadaverine.

### Example 9

### Application of Monoamine Oxidase MAO6^{sh} in Commercially Available Huangjiu

The alcohol content of the commercially available Huangjiu is about 14%-20% vol, the brewing process will produce some organic acids, the pH value of the commercially available Huangjiu is about 3.5-4.5, and the total content of biogenic amines is about 80-150mg/L. The ethanol with a higher concentration and high acidity of the Huangjiu are very unfavorable for the catalytic reaction of enzymes, so the enzymatic control of the content of biogenic amines in the Huangjiu requires more stringent conditions. Specific steps were as follows:

The recombinant monoamine oxidase MAO6^{sh} prepared in Example 3 was subjected to ultrafiltration and concentration to adjust the protein concentration to 200 μg/ml, and then added to the Huangjiu in a ratio of 1:1 for reacting at a room temperature of about 28° C. for 48 h. The control group was commercially available Huangjiu (18% vol) without enzymes, and the total content of biogenic amines in the control group was 136.2 mg·L⁻¹.

The results were shown in Table 1.

TABLE 1
Degradation rates of different biogenic amines by recombinant enzyme

| Biogenic | | | | | | | | |
|---|---|---|---|---|---|---|---|---|
| amine | Tryptamine | Putrescine | Phenylethylamine | Cadaverine | Histamine | Tyramine | Spermidine | Spermine |
| Degradation | 54.07 | 33.76 | 5.78 | 43.78 | 21.44 | 19.31 | 8.51 | 7.75 |
| rate % | | | | | | | | |

The results showed that the commercially available Huangjiu contains 8 types of biogenic amines, the total content of biogenic amines was 136.2 mg·L⁻¹, and tryptamine and cadaverine were the two most important biogenic amines therein. After calculation, the recombinant monoamine oxidase MAO6^{sh} has the degradation rate for total biogenic amines in the commercially available Huangjiu of 25.56%, and has the degradation rates for tryptamine, putrescine and cadaverine of 54.07%, 33.76% and 43.78% respectively.

### Example 10

### Application of Monoamine Oxidase MAO6^{sh} in Commercially Available Low-Salt Soy Sauce

The soy sauce is mainly brewed from soybeans or black beans, wheat or bran, and table salt through processes such as oil making and fermentation. The components of the soy sauce are relatively complex, the range of the total content of biogenic amines in a sample is 41.18-1898.17 mg/L, the salt content of ordinary soy sauce is about 12 g NaCl/100 mL, and the salt content of low-salt soy sauce is about 8 g NaCl/100 mL. The pH is about 4.4-4.6. The NaCl and acidity limit the progress of the enzymatic catalytic reaction.

The recombinant monoamine oxidase MAO6^{sh} prepared in Example 3 was subjected to ultrafiltration and concentration to adjust the protein concentration to 200 ug/ml, and then added to the commercially available low-salt soy sauce for standing at a room temperature (25° C.) for 24-48 h. The results showed that the monoamine oxidase MAO6^{sh} can effectively reduce the content of biogenic amines in the low-salt soy sauce.

### Example 11

### Application of Monoamine Oxidase MAO6^{sh} in Table Vinegar

The table vinegar is a sour seasoning produced by various fermentations, and the content of the acetic acid contained in the table vinegar varies and generally ranges from 5% to 8%. The total amount of biogenic amines in different brands of table vinegar varies greatly, and the total amount of biogenic amines can reach up to 229.98 mg/L.

The recombinant monoamine oxidase MAO6^{sh} prepared in Example 3 was subjected to ultrafiltration and concentration to adjust the protein concentration to 200 μg/ml, and then added to the commercially available table vinegar for standing at a room temperature (25° C.) for 24-48 h. The results showed that the monoamine oxidase MAO6^{sh} can effectively reduce the content of biogenic amines in the table vinegar.

### Example 12

### Application of Monoamine Oxidase MAO6^{sh} in Fermented Sausage

65-80% of lean meat and 20-35% of fat meat were taken by mass and cleaned, and bones, tendons, myolemma, lymph nodes, blood vessels, lesions and injury sites were removed. The fat meat and the lean meat were separated and cut into 4-5 cm meat pieces. The lean meat and about 5-8% of flake ice were placed in a chopper and chopped for 1-3 min. Based on the mass of pork, 0.01-0.15% of sodium nitrite, 2-3% of table salt, 0.2-0.3% of compound phosphate, and 0.05-0.06% of sodium ascorbate were added. Spices, pepper, garlic, chili, and nutmeg were 0.2-0.3% of the raw meat. The recombinant monoamine oxidase MAO6^{sh} prepared in Example 3 was subjected to ultrafiltration and concentration to adjust the protein concentration to 200 μg/ml, and then added to the sausage raw material for chopping for 1-2 min, and then, the fat meat and about 5-8% of flake ice were added for chopping for 4-6 min. The sausage was pickled and then poured into the sausage casing. The sausage poured into the sausage casing was pickled at 4° C. for 12 h, then heated to 30° C., fermented until the pH decreased to about 5.1, and matured at 14-16° C. for 1-10 d. Compared with the fermented sausage without the monoamine oxidase MAO6^{sh}, the results showed that the monoamine oxidase MAO6^{sh} can effectively reduce the content of biogenic amines in the fermented sausage.

At present, there is almost no research on degradation of biogenic amines in fermented food systems by the monoamine oxidase. The monoamine oxidase provided in the present disclosure and the method for degrading biogenic amines in the food industry lay a technical foundation of the monoamine oxidase for catalytic degradation of biogenic amines in relatively harsh systems such as Huangjiu, cooking wine and fruit wine containing alcohols and acids, and have greater potential for other systems with higher content of tryptamine and cadaverine.

Although the present disclosure has been disclosed above with preferred examples, it is not intended to limit the present disclosure. Anyone familiar with this technology can make various changes and modifications without departing from the spirit and scope of the present disclosure. Therefore, the scope of protection of the present disclosure should be defined by the claims.

What is claimed is:

1. A genetically engineered bacterium, wherein Escherichia coli BL21 (DE3) is used as a host to express a monoamine oxidase shown in SEQ ID NO:1.

2. The genetically engineered bacterium according to claim 1, wherein E. coli BL21 (DE3) is used as a host.

3. The genetically engineered bacterium according to claim 2, wherein pET28a(+) is used as an expression vector to express the monoamine oxidase gene shown in SEQ ID NO:3.

4. A method for preparing a monoamine oxidase, wherein the genetically engineered bacterium according to claim 1 is cultured in a culture medium for a period of time, and the monoamine oxidase is collected.

5. The method according to claim 4, wherein the method involves collecting bacterial cells from a cell culture fluid, and crushing the cells to obtain a crude enzyme solution containing the monoamine oxidase.

6. The method according to claim 5, wherein the method further involves purifying the crude enzyme solution.

7. A method for reducing a content of biogenic amines in food, wherein a monoamine oxidase shown in SEQ ID NO:1 is added to food to degrade the biogenic amines in the food.

8. The method according to claim 7, wherein the food is fermented food; and the fermented food comprises fermented vegetables and alcoholic beverages.

9. The method according to claim 8, wherein the fermented food is table vinegar, soy sauce, Huangjiu, or fermented sausage.

10. The method according to claim 8, wherein the biogenic amine comprises one or more of tryptamine, phenylethylamine, putrescine, cadaverine, histamine, tyramine, spermidine, and spermine.

11. The method according to claim 7, wherein the method comprises adding the monoamine oxidase obtained by fermentation of a genetically engineered bacterium that expresses the monoamine oxidase to the food and allowing the mixture to react at 20-25° C. for 24-48 hours.

12. The method according to claim 7, wherein the method comprises adding the monoamine oxidase to Huangjiu and allowing the mixture to react at a room temperature of 25-28° C. for 24-48 hours.


SEQUENCE LISTING (5 sequences)

SEQ ID NO: 1 - protein, 433 aa, Saccharopolyspora hirsuta
MDSYDVVVIGAGFAGLVAARELALRGHAVVVLEARDRIGGRTWTDERLGRRLEMGGTWVHWLQPHVWSEITRYDQRIEPSPSSQAVHWVAGGSAHRGSPEEFDALISPGMDQLARDSRAWFPLPYEPLHRADLDQVDHLSVADYFAGLDLNPVEREVTEGVWAEHFNGPAAVSGYTQAMRWCAAASGNWQLLHQATSGYRLTGGTAALAGAIAADGGAELHLGSVVASVSQDSGGATVSTTDGTSVRARRVICTLPLNVLGTVDFQPALPATKLAASAERTASQGLKTWIRVRGEVAPFTAYAPDSHDLTFVRPEYTVEGDTLLVAFGTRAADLAPDDVDGVARALRVWRDDLEVLEVSGHDWMTDDFSRETWPMQRPEQLTRYLAALQEPHGVVRFAGSDLATGWAGFIDGAVESGLRASRAVQDALRGPRG

SEQ ID NO: 2 - DNA, 1314 bp, synthetic construct
  source 1..1314: mol_type = other DNA
gctagcatggactcctacgacgtcgtcgtcatcggtgccggattcgccgggctcgtggcggcccgcgagctggccctgcgcgggcacgcggtggtggtgctcgaggcccgcgaccgcatcggcgggcggacctggaccgacgagcgcctgggtcggcggctggagatgggcggcacctgggtccactggctgcagccgcacgtctggagcgagatcacccgctacgaccagcgcatcgagccgagtccgagctcgcaggcggtccactgggtcgccgggggcagcgcgcaccgcggcagcccggaggagttcgacgccctgatctcacctggcatggaccagttggcgcgggacagccgcgcctggttcccactcccctacgaaccgctgcaccgggccgacctggaccaggtcgaccacctctcggtcgccgactacttcgcggggctcgacctaaacccggtggagcgggaggtgaccgagggcgtgtgggcggagcacttcaacggcccggccgcggtctccgggtacacccaggccatgcgctggtgcgccgcggcctccggcaactggcagctgctgcaccaggccacctcgggttaccggctgaccggcggcaccgcggcgctggccggggctatcgccgccgacggaggcgcggagctccacctcggctcggtggtcgccagcgtctcgcaggactccggcggcgcgacggtcagcaccaccgacggcaccagcgtccgggcccgccgggtgatctgcacgctgccgctcaacgtgctgggcacggtcgacttccagcccgcgctccccgcgaccaagctggccgccagtgccgagcgcaccgcttcgcaggggctgaagacctggatccgggtgcgcggcgaggtcgcgccgttcaccgcctacgcgcccgacagccacgatctgaccttcgtgcgccccgagtacacagtggagggcgacacgctgctggtcgccttcggcacccgcgctgccgacctcgcccccgacgacgtcgacggggtggcccgggcgctgcgggtgtggcgcgacgacctggaggtgctcgaggtcagcggccacgactggatgaccgacgacttctcccgcgagacctggcccatgcaacggccggagcagttgacccggtacctggccgcgctgcaggaaccgcacggggtcgtgcgcttcgccgggtcggacctggccaccggctgggcgggcttcatcgacggcgcggtcgagagcggcctgcgcgcctccagggccgtgcaggacgccctgcgcggcccgcgaggctgaaagctt

SEQ ID NO: 3 - DNA, 1302 bp, synthetic construct
  source 1..1302: mol_type = other DNA
atggactcctacgacgtcgtcgtcatcggtgccggattcgccgggctcgtggcggcccgcgagctggccctgcgcgggcacgcggtggtggtgctcgaggcccgcgaccgcatcggcgggcggacctggaccgacgagcgcctgggtcggcggctggagatgggcggcacctgggtccactggctgcagccgcacgtctggagcgagatcacccgctacgaccagcgcatcgagccgagtccgagctcgcaggcggtccactgggtcgccgggggcagcgcgcaccgcggcagcccggaggagttcgacgccctgatctcacctggcatggaccagttggcgcgggacagccgcgcctggttcccactcccctacgaaccgctgcaccgggccgacctggaccaggtcgaccacctctcggtcgccgactacttcgcggggctcgacctaaacccggtggagcgggaggtgaccgagggcgtgtgggcggagcacttcaacggcccggccgcggtctccgggtacacccaggccatgcgctggtgcgccgcggcctccggcaactggcagctgctgcaccaggccacctcgggttaccggctgaccggcggcaccgcggcgctggccggggctatcgccgccgacggaggcgcggagctccacctcggctcggtggtcgccagcgtctcgcaggactccggcggcgcgacggtcagcaccaccgacggcaccagcgtccgggcccgccgggtgatctgcacgctgccgctcaacgtgctgggcacggtcgacttccagcccgcgctccccgcgaccaagctggccgccagtgccgagcgcaccgcttcgcaggggctgaagacctggatccgggtgcgcggcgaggtcgcgccgttcaccgcctacgcgcccgacagccacgatctgaccttcgtgcgccccgagtacacagtggagggcgacacgctgctggtcgccttcggcacccgcgctgccgacctcgcccccgacgacgtcgacggggtggcccgggcgctgcgggtgtggcgcgacgacctggaggtgctcgaggtcagcggccacgactggatgaccgacgacttctcccgcgagacctggcccatgcaacggccggagcagttgacccggtacctggccgcgctgcaggaaccgcacggggtcgtgcgcttcgccgggtcggacctggccaccggctgggcgggcttcatcgacggcgcggtcgagagcggcctgcgcgcctccagggccgtgcaggacgccctgcgcggcccgcgaggctga

SEQ ID NO: 4 - DNA, 53 bp, synthetic construct
  source 1..53: mol_type = other DNA
ctagctagcatggactcctwcgacgtcgtsgtcatcggtgccggrttcgccgg

SEQ ID NO: 5 - DNA, 65 bp, synthetic construct
  source 1..65: mol_type = other DNA
caagctttcagcctcgsgggccgcgcagggcgtcctgcacggccctggaggcgcgcaggccgctc